(19) United States
(12) Patent Application Publication
(10) Pub. No.: US 2010/0305339 A1
(43) Pub. Date: Dec. 2, 2010

(54) METHOD FOR PRODUCING LACTONES FROM DIOLS

(75) Inventors: Rolf Pinkos, Bad Duerkheim (DE); Daniel Breuninger, Bobenheim-Roxheim (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/446,068

(22) PCT Filed: Oct. 12, 2007
(86) PCT No.: PCT/EP07/60896

§ 371 Date: Apr. 17, 2009

(30) Foreign Application Priority Data
Oct. 18, 2006 (EP) 06122524.9

(51) Int. Cl. C07D 309/12; C07D 313/04

(52) U.S. Cl. 549/266; 549/273

(57) ABSTRACT

The present invention provides a process for preparing lactones from optionally substituted, saturated aliphatic diols having from five to 20 carbon atoms between the two ring-closing hydroxyl groups by catalytic dehydrogenation and cyclization in the liquid phase over at least one catalyst.

The present invention relates to a process for preparing lactones having a ring size of at least 6 from optionally substituted, saturated aliphatic diols having from five to 20 carbon atoms between the two hydroxyl groups by catalytic dehydrogenation and cyclization in the liquid phase over at least one catalyst.

The conversion of diols to the corresponding lactones is known. The most prominent example is the dehydrogenation of 1,4-butanediol to gamma-butyrolactone in the gas phase at standard pressure in very high yields, which is described, for example, in K. Weissermel and H.-J. Arpe, “Industrielle Organische Chemie” [Industrial Organic Chemistry], WILEY-VCH Verlag GmbH, 69469 Weinheim, 5th edition 1998, page 114.

When attempts are made to apply this technology to lactones with larger ring size, the reaction temperatures have to be raised or vacuum has to be applied or a large amount of hydrogen has to be used as a carrier gas owing to the higher boiling points of the diols, which has an adverse effect on the achievable yields or means increased complexity in the reaction. For example, in the dehydrocyclization of 1,6-hexanediol in the gas phase according to working example 2 of U.S. Pat. No. 3,317,563, a molar ratio of hydrogen to 1,6-hexanediol of 20 to 1 is employed at 250° C. However, only a yield of 50% is achieved at a selectivity of 82%. S. Oka also describes, in the Bulletin Chem. Soc. Japan 35 (1962), p. 562-566, for the synthesis of ε-caprolactone from 1,6-hexanediol, a yield of 51% with a selectivity of approx. 65% at pressures of from 5 to 10 torr and temperatures of from 210 to 220° C. U.S. Pat. No. 3,317,563 warns, in column 1, against the performance of the dehydrocyclization in the liquid phase, since it results in the formation of considerable amounts of polymeric lactones there.

In addition to the abovementioned catalytic processes, processes in which at least stochiometic amounts of oxidizing agents are consumed are also known. These processes are not an option for an industrial scale reaction, since they are uneconomic owing to the costs of the oxidizing agent alone.

It is an object of the present invention to provide a very simple, economically viable process with which saturated diols having from five to 20 carbon atoms between the two ring-closing hydroxyl groups can be converted to the corresponding lactones without the aforementioned disadvantages occurring.

This object is achieved by a process for preparing lactones from optionally substituted, saturated aliphatic diols having from five to 20 carbon atoms between the two ring-closing hydroxyl groups by catalytic dehydrogenation and cyclization in the liquid phase over at least one catalyst.

The process according to the invention permits selective conversion of the diol to the corresponding lactone with good yield. The lactones formed are sought-after starting materials for the preparation of polyesters, for example for coatings. A particularly preferred lactone for these uses is ε-caprolactone.

The process according to the invention is suitable for linear diols or saturated diols which are branched by the substitution which may be present, having from at least five to 20 carbon atoms between the two ring-closing hydroxyl groups, for example 1,5-pentanediol, 1,6-hexanediol, 1,7-heptanediol, 1,8-octanediol, 1,10-decanediol and 1,12-dodecanediol. The diols useable in accordance with the invention may be substituted by one or more C₁- to C₁₀-alkyl, C₅- to C₁₂-aryl and/or C₁- to C₁₀-alkoxy groups. Preference is given to linear diols having from five to 12 carbon atoms; particular preference is given to 1,5-pentanediol and 1,6-hexanediol.

The process according to the invention for catalytic dehydrogenation and cyclization (dehydrocyclization) is a transition metal-catalyzed process in which the diol is converted to the hydroxycarboxylic acid and then cyclized in the liquid phase.

According to the invention, in a two-stage variant of the process (variant A), at least one catalyst is used for the dehydrogenation and at least one catalyst for the cyclization, and the catalyst for the two reaction steps can be the same. According to the invention, a two-stage process is understood to mean the performance of the process in two spatially distinguishable reaction chambers, or reaction chambers separated from one another. These distinguishable or mutually separated reaction chambers may be reactors of the same type with suitable separating devices in order to spatially separate the reaction zone for the dehydrogenation from the reaction zone of the cyclization, or be different reactors. In this application, different reactors are understood to mean either different reactor types or reactors of the same type which differ, for example, by their geometry, for example their volume and/or their cross section and/or by the reaction conditions in the reactors.

Catalysts for the inventive dehydrogenation may be homogeneous or heterogeneous, metallic catalysts, where the metal may be present in elemental form or in the form of a compound, for example as an oxide, hydride, halide, salt of a carboxylic acid or complex.

The useable catalysts preferably comprise at least one metal of transition group 7, 8, 9, 10, 11 or 12 of the Periodic Table of the elements or one metal from these groups. More preferably, the catalysts useable in accordance with the invention comprise at least one element selected from the group consisting of Re, Fe, Ru, Co, Rh, If, Ni, Pd, Pt, Cu and Au. Especially preferably, the catalysts useable in accordance with the invention comprise at least one element selected from the group consisting of Ni, Pd, Pt, Ru and Cu. More preferably, the catalysts useable in accordance with the invention comprise Pd, Pt, Ru or Cu. The catalysts used in accordance with the invention are preferably chromium-free.

A suitable catalyst for the inventive dehydrogenation is in particular at least one heterogeneous catalyst, and it is possible for at least one of the abovementioned metals to be used as the metal as such, as a Raney catalyst and/or applied to a customary support. When two or more metals are used, they may be present separately or as an alloy. It is possible here to use at least one metal as such and at least one metal as a Raney catalyst, or at least one metal as such and at least one other metal applied to at least one support, or at least one metal as a Raney catalyst and at least one other metal applied to at least one support, or at least one metal as such and at least one other metal as a Raney catalyst and at least one other metal applied to at least one support.

In addition, the catalysts used may also be so-called precipitation catalysts. Such catalysts can be prepared by precipitating their catalytically active components from their salt solutions, in particular from the solutions of their nitrates and/or acetates, for example by adding solutions of alkali metal and/or alkaline earth metal hydroxide and/or carbonate solutions, for example sparingly soluble hydroxides, oxide hydrates, basic salts or carbonates, then drying the resulting precipitates and then converting them by calcination at generally from 300 to 700° C., in particular from 400 to 600° C., to the corresponding oxides, mixed oxides and/or mixed-valency oxides, which are reduced to the metals in question and/or oxidic compounds of lower oxidation state by a treatment with hydrogen or with hydrogen-comprising gases in the range of generally from 50 to 700° C., in particular from 100 to 400° C., and converted to the actual catalytically active form. Reduction is generally continued until no further water is formed. In the preparation of precipitation catalysts which comprise a support material, the catalytically active components can be precipitated in the presence of the support material in question.

The catalytically active components may advantageously be precipitated simultaneously with the support material from the salt solutions in question.

Preference is given to using dehydrogenation catalysts in which the metals or metal compounds which catalyze the dehydrogenation have been applied on a support material, known as supported catalysts.

The way in which the catalytically active metal or the metal compound is applied to the support is generally uncritical and can be accomplished in a variety of different ways. The catalytically active metals may be applied to these support materials, for example, by impregnation with solutions or suspensions of the salts or oxides of the elements in question, drying and subsequent reduction of the metal compounds to the metals in question or compounds of lower oxidation state by means of a reducing agent, preferably with hydrogen or complex hydrides. Another means of applying the catalytically active metals to these supports consists in impregnating the supports with solutions of thermally readily decomposable salts, for example with nitrates or thermally readily decomposable complexes, for example carbonyl or hydrido complexes of the catalytically active metals, and heating the support thus impregnated to temperatures in the range from 300 to 600° C. for thermal decomposition of the adsorbed metal compounds. This thermal decomposition is preferably undertaken under a protective gas atmosphere. Suitable protective gases are, for example, nitrogen, carbon dioxide, hydrogen or the noble gases. In addition, the catalytically active metals may be deposited on the catalyst support by vapor deposition or by flame-spraying. The content in these supported catalysts of the catalytically active metals is in principle uncritical for the success of the process according to the invention. In general, higher contents of catalytically active metals in these supported catalysts lead to higher space-time yields than lower contents. In general, supported catalysts whose content of catalytically active metals is in the range from 0.1 to 90% by weight, preferably in the range from 0.5 to 40% by weight, based on the total weight of the catalyst, are used. Since these content data are based on the overall catalyst including support material, but the different support materials have very different specific weights and specific surface areas, it is also conceivable that this content data may go below or above these figures without this having any disadvantageous effect on the result of the process according to the invention. It will be appreciated that it is also possible for a plurality of the catalytically active metals to be applied on the particular support material. In addition, the catalytically active metals can be applied to the support, for example, by the process of DE-A 25 19 817, EP 1 477 219 A1 or EP 0 285 420 A1. In the catalysts according to the aforementioned documents, the catalytically active metals are present as alloys which are obtained by thermal treatment and/or reduction of the, for example, by impregnation of the support material with a salt or complex of the aforementioned metals.

The support materials used may generally be the oxides of zinc, of aluminum and of titanium, zirconium dioxide, silicon dioxide, lanthanum oxide, aluminas, for example montmorillonites, silicates, for example magnesium silicates or aluminum silicates, zeolites, for example of the ZSM-5 or ZSM-10 structure types, or activated carbon. Preferred support materials are aluminum oxides, titanium dioxides, silicon dioxide, zirconium dioxide and activated carbon. It will be appreciated that it is also possible for mixtures of different support materials to serve as the support for catalysts useable in the process according to the invention. It is also possible for alkali metal and/or alkaline earth metal compounds, preferably as oxides, to be present as additives for the controlled adjustment of the acidic, in particular basic, properties of the catalyst. Possible catalysts are also those which comprise zinc oxide or zirconium oxide as the active component.

According to the invention, very particularly preferred catalysts are those which comprise Cu, Pt, Ru and/or Pd and are applied on a support. Very preferred supports are or comprise activated carbon, aluminum oxide, titanium dioxide, lanthanum oxide and/or silicon dioxide.

Heterogeneous catalysts are, if necessary, generally activated before use, preferably with hydrogen. The methods for this purpose are known to those skilled in the art.

Heterogeneous catalysts used in accordance with the invention are generally activated in a manner known per se before use in the inventive dehydrogenation. The activation is preferably effected with hydrogen. Both the activation of the precipitation catalysts and of the supported catalysts can also be effected in situ at the start of the reaction by means of hydrogen. Preference is given to activating these catalysts separately before use.

Also suitable are homogeneous catalysts comprising at least one element of transition group 8, 9 or 10. More preferred are homogeneous catalysts which comprise Ru, Rh, Ir and/or Ni.

When compounds of the aforementioned elements are used, suitable examples are salts such as halides, oxides, nitrates, sulfates and carbonates, alkoxides and aryloxides, carboxylates, acetylacetonates, acetates of the particular metal. It is also possible for these salts to be modified with complexing ligands. The compounds utilized in accordance with the invention preferably comprise exclusively complexing ligands. The ligands may be oxygen-, sulfur-, nitrogen- or phosphorus-containing compounds and be present in charged or uncharged form. Examples of these ligand types are CO, CS, optionally organyl-substituted amino ligands, optionally organyl-substituted phosphine ligands such as triphenylphosphine (TPP), chelate ligands such as 1,1,1-tris(diphenylphosphinomethyl)ethane, and alkyl, aryl, allyl, cyclopentadienyl and olefin ligands.

For example, mention should be made here, for instance, of RhCl(TPP)₃ or Ru₄H₄(CO)₁₂. Particular preference is given to those homogeneous catalysts which comprise Ru. For example, homogeneous catalysts as described in U.S. Pat. No. 5,180,870, U.S. Pat. No. 5,321,176, U.S. Pat. No. 5,177,278, U.S. Pat. No. 3,804,914, U.S. Pat. No. 5,210,349, U.S. Pat. No. 5,128,296, U.S. Pat. No. 316,917 and in D. R. Fahey in J. Org. Chem. 38 (1973) p. 80-87, whose disclosure content in this regard is incorporated fully into the context of the present application, are used. Examples of such preferred homogeneous catalysts include (TPP)₂(CO)₃Ru, [Ru(CO)₄]₃, (TPP)₂Ru(CO)₂Cl₂, (TPP)₃(CO)RuH₂, (TPP)₂(CO)₂RuH₂, (TPP)₂(CO)₂RuClH or (TPP)₃(CO)RuCl₂.

For the dehydrogenation, preference is given to using at least one heterogeneous catalyst which can be used, for example, as a suspension catalyst and/or as a fixed bed catalyst.

When the inventive dehydrogenation is performed with at least one suspension catalyst, the reaction is effected preferably in at least one stirred reactor or in at least one bubble column or in at least one packed bubble column or in a combination of two or more identical or different reactors.

The suspension catalyst used in the inventive dehydrogenation is, on completion of reaction, preferably removed by at least one filtration step. The suspension catalyst removed can be recycled into the dehydrogenation or be sent to any other process. It is equally possible to work up the catalyst in order, for example, to recover the metal present in the catalyst.

The inventive dehydrogenation can additionally be performed with at least one fixed bed catalyst. In this method, preference is given to using at least one tubular reactor, for example at least one shaft reactor and/or at least one tube bundle reactor, and an individual reactor may be operated in liquid phase or trickle mode. When two or more reactors are used, at least one may be operated in liquid phase mode and at least one in trickle mode. A fixed bed catalyst can additionally be introduced in a distillation column in the form of a packing or as part of a packing. When the catalyst itself serves as a packing, preference is given to applying the catalyst as a coating, for example to a metal fabric.

When the catalyst used in the dehydrogenation is a homogeneous catalyst, in the context of the present invention, it is preferably also conducted into the next reaction step and then recycled into the dehydrogenation either together with the diol or, if appropriate, after removal and purification. The homogeneous and heterogeneous catalysts used for the inventive dehydrogenation may be regenerated in a manner known per se by suitable processes and reused.

For variant A of the process according to the invention, the inventive dehydrogenation is performed generally at from 0.01 to 100 bar (absolute), preferably from 0.05 to 20 bar (absolute), more preferably from 0.07 to 5 bar (absolute), especially preferably from 0.1 to 2 bar (absolute), and at temperatures of 50-350° C., preferably of 100-280° C., more preferably of 150-250° C. The reaction conditions are selected such that, with the exception of the hydrogen formed, the diol and the reaction products remain quite predominantly in the liquid phase. The catalytic dehydrogenation is preferably performed with exclusion of oxygen.

The hydrogen released in the dehydrogenation is removed from the reaction mixture. It may be sufficient if it leaves the liquid phase spontaneously and collects in the gas phase. However, preference is given to continually removing the hydrogen, especially when the process according to the invention is performed continuously. This can be achieved, for example, by sucking the hydrogen out by means of a vacuum, performing the reaction at elevated pressure and decompressing the hydrogen to ambient pressure, or by stripping it out by means of inert gases, for example with nitrogen or argon. It is also possible in principle to remove the hydrogen by a reaction which consumes it, for example by simultaneously performing a hydrogenation, for example of a double bond.

The reaction mixture formed in the inventive dehydrogenation comprises generally, in addition to a small amount (<5% by weight) of free lactone and the ester of the diol and the hydroxycarboxylic acid, also free diol, small amounts of oligomeric esters formed from hydroxycarboxylic acids and diol, and also intermediates, for example hemiacetals. The reaction mixture preferably comprises less than 1% by weight of dicarboxylic acid products, more preferably less than 0.5%, most preferably less than 0.1%. Dicarboxylic acids and their esters can form when the starting material is a diol having two primary OH groups and both sides are dehydrogenated. In the process according to the invention, the conversion of the diol is preferably restricted to less than 75%; the conversion is more preferably between 10 and 50%. The process can also be performed at conversions lower than 10%.

In variant A of the process according to the invention, the reaction mixture obtained in the inventive dehydrogenation is converted to the lactone in a second stage (cyclization) in the liquid phase over at least one catalyst.

Suitable catalysts for the cyclization are acidic or basic catalysts which may be present in homogeneously dissolved or heterogeneous form. Suitable catalysts are alkali metal and alkaline earth metal hydroxides, oxides, carbonates, alkoxides or carboxylates, inorganic acids such as sulfuric acid or phosphoric acid, organic acids such as sulfonic acids and salts of the aforementioned acids, Lewis acids or bases, preferably from group 3 to 15 of the Periodic Table of the elements. Particular preference is given to using Lewis acids or bases based on aluminum, tin, antimony, zirconium or titanium, for example AICl₃, Al(OR)₃, where R is a C₁- to C₂₀-alkyl radical, SbCl₅, SnCl₄, ZrCl₄, Zr(OR)₄, TiCl₄, Ti(OR)₄. Especially preferred are Lewis acids or bases of titanium, such as tetraisopropyl titanate, tetrabutyl titanate or mixtures thereof.

The concentration of the catalysts present in homogeneously dissolved form is from 10 to 20 000 ppm, preferably from 100 to 5000 ppm, more preferably from 300 to 3000 ppm. The cyclization is performed typically at from 100 to 400° C., preferably from 150 to 300° C., more preferably from 170 to 250° C. The reaction pressure is between 1 and 2000 mbar (absolute), preferably between 10 and 1013 mbar (absolute), more preferably between 20 and 1013 mbar (absolute).

In a particularly preferred embodiment of the process according to the invention of variant A, the dehydrogenation and the cyclization are performed with distillative removal of the lactone formed, especially preferably in a reactor, but in separate reaction zones. In this case, preferably only the lactone is distilled off. However, it is also possible to distill off the diol, or a portion thereof, together with the lactone, in which case the diol has to be removed from the lactone in a further distillation step. Diol recovered from the cyclization is generally recycled into the dehydrogenation, if appropriate after purification.

It is possible for the inventive catalysts of the two reaction steps to be present together or spatially separately in one reactor. More preferably, the two catalysts are present in a distillation column in which the two reaction steps proceed in succession and lactone is distilled off. In this preferred embodiment, the dehydrogenation catalyst may be installed on the trays in the stripping section and/or rectifying section, and the catalyst for the cyclization may be present in the bottom of the column. In this particular embodiment in a distillation column, vaporous diol passes into the stripping section and/or rectifying section of the column, and diol condenses and is reacted partly over the dehydrogenation catalyst as it refluxes, while lactone which has already formed is removed via the top. The converted diol passes, as a high-boiling hydroxycarboxylic ester, into the bottom of the column in which the lactone formation proceeds. The lactone formed passes back into the column in vaporous form together with diol which is yet to be converted. Unconverted diol can be removed from the reaction mixture via a side draw of the column.

In a further particular embodiment of the process according to the invention, the dehydrogenation and the cyclization are performed in one reaction stage (one-stage: variant B) over at least one catalyst. In this case, the catalysts and the reaction conditions are selected such that the desired dehydrogenation and the cyclization to the lactone are achieved over at least one catalyst in the same working step. In variant B, preference is given to using the same catalyst for the dehydrogenation and the cyclization. The distillative removal of the lactone formed is absolutely necessary in this embodiment. Suitable catalysts for the process according to the invention (in variant B) are the catalysts described for the dehydrogenation under variant A, which may, if appropriate, also comprise those of the cyclization stage. In variant B, the process according to the invention is performed in the liquid phase at from 1 to 2000 mbar (absolute), preferably between 10 and 1013 mbar (absolute), more preferably between 20 and 1013 mbar (absolute), and temperatures of from 100 to 400° C., preferably from 150 to 300° C., more preferably from 190 to 250° C., with the distillative removal of the lactone. The catalysts used may be present in heterogeneous and/or homogeneously dissolved form.

The process according to the invention in variant B is preferably performed with at least one suspended catalyst present in heterogeneous form. This catalyst is preferably present in the bottom of a column, the mixing being ensured by stirring and/or by pumped circulation.

In the inventive dehydrogenation, the conversion of the diol is preferably restricted to less than 75%; the conversion is more preferably between 10 and 50%. The process may, though, also be performed at conversions lower than 10%. In the preferred continuous performance of the process according to the invention, this means that at least 75% of the diol is present in the reaction mixture at least in the cyclization step. Caused by recycling of the diol and/or continuous feeding of diol, the overall conversion in the process is higher than 25%, preferably >90%, more preferably >95%.

The different process variants can be operated batchwise, but preferably continuously in an industrial scale application. The performance of the process according to the invention with complete or partial recycling of the diol used is particularly economically viable.

The process according to the invention will be illustrated in detail with reference to the example which follows.

### EXAMPLES

### Example 1

20 g of 1,6-hexanediol, 3.2 g of a hydrogen-activated Cu catalyst which, in the oxidic state, consists of approx. 60% copper oxide and approx. 40% aluminum oxide were introduced into a glass flask with attached column. After the pressure had been lowered to 100 mbar (absolute) and the bottom had been heated to 190° C., 13 g of a mixture which, as well as the ester formed from hexanediol and 6-hydroxycaproic acid, contained 50% 1,6-hexanediol and 30% ε-caprolactone were distilled within 2 hours. In the bottom of the column, approx. 15% 1,6-hexanediol, approx. 5% caprolactone and small proportions of dimeric and oligomeric esters formed from hexanediol and 6-hydroxycaproic acid were also found in the bottom of the column after 2 hours. The caprolactone yield was approx. 20%, the selectivity >90%.

### Example 2

20 g of 1,6-hexanediol, 0.3 g of ruthenium trisacetylacetonate and 0.7 g of 1,1,1-tris(diphenylphosphinomethyl)ethane were heated in an autoclave to 150° C. at 150 bar of hydrogen pressure for 6 hours. Subsequently, the reaction mixture was distilled at 200 mbar and 190° C. A mixture of 60% hexanediol, 8% caprolactone and 6% 6-hydroxyhexanal or its cyclic hemiacetal, and also further products which were not analyzed further, was obtained. The yield of caprolactone was approx. 50%, the selectivity >95%.

### Example 3

50 g of hexanediol, 0.01 g of tetraisopropyl titanate were initially charged in a glass flask with attached column. 12 g of a catalyst which contained 0.1% PD and 0.1% Cu on Al₂O₃ were installed into the attached column. The aluminum oxide was present on a mesh fabric; this fabric together with the catalytically active constituents was present as a coil in the column. After the pressure had been reduced to 50 mbar, the system was heated to 135° C. for distillation. In the distillate (40 g), caprolactone (approx. 2%) was present in addition to hexanediol (approx. 98%). In the bottom, hexanediol (approx. 95%), caprolactone (approx. 3%) and esters formed from hexanediol and 6-hydroxycaproic acid were found. The yield of caprolactone was approx. 2% with selectivities of >95%.

### Example 4

150 g of 1,6-hexanediol were initially charged in a glass flask and 20 g of a hydrogen-activated Cu catalyst which, in the oxidic state, had consisted of approx. 60% copper oxide and approx. 40% aluminum oxide were installed in the attached column. The column contents were heated to 200° C. for 4 hours and to 250° C. for 2 hours and, after cooling, admixed with 2.9 g of titanium tetrabutoxide. After the pressure had been reduced to 30 mbar, a distillate which comprised caprolactone (33%) in addition to 1,6-hexanediol (54%) was collected at 170° C. In the bottoms, hexanediol (41%), caprolactone (12%) and esters formed from hexanediol and 6-hydroxycaproic acid (approx. 28%) were found.

### Example 5

100 g of 1,5-pentanediol, 3 g of a hydrogen-activated Cu catalyst which, in the oxidic state, consisted of approx. 60% copper oxide and approx. 40% aluminum oxide were introduced into a glass flask with attached column. After the pressure had been lowered to 100 mbar (absolute) and the bottom had been heated to 190° C., 74.3 g of a mixture of 60.6 g of 1,5-pentanediol and 9.6 g of valerolactone distilled over within 2 hours. In the bottom of the column, 1,5-pentanediol and esters of 5-hydroxypentanoic acid remained. Valerolactone was obtained in 22% yield; the selectivity was >95%.

1. A process for preparing lactones, comprising catalytically dehydrogenating and cyclizing in the liquid phase over at least one catalyst, optionally substituted, saturated aliphatic diols, wherein said diols have from five to 20 carbon atoms between the two ring-closing hydroxyl groups.

2. The process according to claim 1, wherein the carbon chain of the saturated aliphatic diols is substituted by at least one of C₁- to C₁₀-alkyl radical, C5- to C₁₂-aryl radical, and C₁- to C₁₀-alkoxy radical.

3. The process according to claim 1, wherein the catalyst is a heterogeneous catalyst, a homogeneous catalyst or a combination thereof.

4. The process according to claim 3, wherein the catalyst is a heterogeneous catalyst comprising at least one metal selected from the group consisting of transition groups 7, 8, 9, 10 and 12 of the Periodic Table of the elements for the dehydrogenation.

5. The process according to claim 4, wherein the metal of the heterogeneous dehydrogenation catalyst is selected from the group consisting of Re, Fe, Ru, Co, Rh, Ir, Ni, Pd, Pt and Cu.

6. The process according to claim 3, wherein the catalyst is a homogeneous dehydrogenation catalyst comprising at least one metal selected from the group consisting of transition groups 8, 9, and 10 of the Periodic Table of the elements.

7. The process according to claim 6, wherein the homogeneous dehydrogenation catalyst used is at least one of a salt and a compound of the particular metal comprising one or more oxygen-, sulfur-, nitrogen- or phosphorus-containing complexing ligands.

8. The process according to claim 1, wherein the cyclization catalyst is alkali metal and alkaline earth metal hydroxides, oxides, carbonates, alkoxylates and carboxylates, inorganic or organic acids and salts thereof, or Lewis acids or bases of the metals of groups 3 to 15 of the Periodic Table of the elements.

9. The process according to claim 1, wherein the reaction is effected with distillative removal of the lactone formed.

10. The process according to claim 1, wherein the reaction is effected in a column.

11. The process according to claim 1, wherein the catalyst is a separate catalyst for the catalytic dehydrogenation, and for the subsequent catalytic cyclization respectively.

12. The process according to claim 1, wherein the catalyst a common catalyst for catalytic dehydrogenation and for cyclization.

13. The process according to claim 1, comprising performing a two-stage process wherein the dehydrogenation is performed at from 0.01 to 100 bar and at temperatures from 50 to 350° C., and the cyclization is performed at from 1 to 2000 mbar and temperatures from 100 to 400° C.

14. The process according to claim 1, comprising performing said process in one stage at from 1 to 2000 mbar and temperatures from 100 to 400° C. with distillative removal of the lactone with at least one catalyst suitable for the dehydrogenation comprising a heterogeneous catalyst, homogeneous catalyst or a combination thereof as a catalyst.

15. The process according to claim 1, wherein the diol used is 1,6-hexanediol.

16. The process according to claim 1, wherein the diol used is 1,5-pentanediol.

17. The process according to claim 4, wherein the metal of said heterogeneous dehydrogenation catalyst is Ni, Pd, Pt or Cu.

18. The process according to claim 6, wherein the homogeneous dehydrogenation catalyst is halides, oxides, nitrates, sulfates, carbonates, alkoxides, aryl oxides, carboxylates, acetylacetonates and acetates of the particular metal, or the compounds of the metal comprising CO, CS, an optionally organyl-substituted amino ligand, an optionally organyl-substituted phosphine ligand, an alkyl, allyl, cyclopentadienyl or olefin ligand.
